(19) United States
(12) Patent Application Publication
(10) Pub. No.: US 2012/0116500 A1
(43) Pub. Date: May 10, 2012

(54) H-Side Branch Stent

(75) Inventors: Yang Soo Jang, Seoul (KR); Myeong Ki Hong, Seoul (KR); Dong Hoon Choi, Seoul (KR)

(73) Assignee: INDUSTRY-ACADEMIC COOPERATION FOUNDATION, YONSEI UNIVERSITY, Seoul (KR)

(21) Appl. No.: 13/320,224

(22) PCT Filed: Nov. 3, 2009
(86) PCT No.: PCT/KR09/06423

§ 371 Date: Jan. 20, 2012

(30) Foreign Application Priority Data
May 13, 2009 (KR) 10-2009-0041769

(51) Int. Cl. A61F 2/82

(52) U.S. Cl. 623/1.35

(57) ABSTRACT

Provided is an H-side branch stent including a first stent having a plurality of cells and a hollow cylindrical shape, at least one bridge having one end connected to a distal end of the first stent, and a second stent having a front end connected to the other end of the bridge, and having a plurality of cells and a hollow cylindrical shape. Accordingly, the H-side branch stent can prevent re-stenosis of the opening of the branch blood vessel since the stent can completely support an inner circumference of an opening of a branch blood vessel in a circumferential direction, and prevent blood flow disturbance because stents are not densely concentrated to a boundary between a main blood vessel and the branch blood vessel and some regions of the stent do not project into the main blood vessel.

### CROSS-REFERENCE TO RELATED APPLICATION

This application claims priority to and the benefit of Korean Patent Application No. 2009-0041769, filed May 13, 2009, the disclosure of which is incorporated herein by reference in its entirety.

### BACKGROUND

1. Field of the Invention

The present invention relates to an H-side branch stent, and more particularly, to an H-side branch stent capable of preventing re-stenosis of the opening of the branch blood vessel since the stent can completely support an inner circumference of an opening of a branch blood vessel in a circumferential direction, preventing blood flow disturbance because stents are not densely concentrated to a boundary between a main blood vessel and the branch blood vessel and a partial region of the stent do not project into the main blood vessel, and completely adhering the stent to an inner wall of the main blood vessel with no gap.

2. Discussion of Related Art

In general, a stent is a medical instrument, which is inserted into a lumen or a blood vessel of a human body to expand the lumen or the blood vessel when the lumen is narrowed to degrade its inherent function due to various diseases occurring in the human body or the blood vessel is narrowed to deteriorate blood circulation.

A coronary artery disease or an ischemic heart disease is a disease generated because a fat-forming element is accumulated on a blood vessel wall of the coronary artery, inflammatory reaction accompanied therewith gradually narrows the lumen of the coronary artery, and blood cannot be sufficiently supplied into the heart muscle due to a size of the narrowed lumen of the coronary artery.

When the blood cannot be sufficiently supplied into the heart muscle, a chest pain, difficulty in breathing and other symptoms occur according to a level of the above-described blood supply. Such a coronary artery disease is represented as clinical signs such as angina, acute myocardial infarction (AMI), sudden death, and so on.

Percutaneous coronary intervention (PCI) is a treatment method of physically expanding the lumen of the coronary artery narrowed due to accumulation of cholesterol onto the blood vessel wall using a balloon catheter or a stent. However, the PCI using the balloon catheter may cause typical complications such as acute closure or dissection.

While the treatment method using the stent can prevent the acute closure or dissection, which may be caused by the treatment method using the balloon catheter, the stent surgery cannot contribute to reducing the re-stenosis in a lesion of a branch of the coronary artery.

FIG. 1 is a concept view showing a lesion N of a branch of the coronary artery, in which stenosis occurs at a boundary of a proximal portion 1a and a distal portion 1b of a main blood vessel 1 and a branch blood vessel 2.

In order to treat an opening of the branch blood vessel 2 showing diagnosis of stenosis, the opening of the branch blood vessel 2 must be positioned such that an inner circumference of the opening is securely supported by a drug- eluting stent in a circumferential direction.

FIGS. 2 and 3 are concept views for explaining a surgically operated state of a conventional stent used in a lesion of the branch blood vessel of the coronary artery. FIG. 2 shows that the stent S is disposed at a partial region of an inner circumference of the branch blood vessel 2 so that the opening of the branch blood vessel 2 cannot be completely surrounded by the stent S, and FIG. 3 shows that the stent S completely surrounds the opening of the branch blood vessel 2 but a partial region C of the stent projects into a space in the main blood vessel 1.

When the stent S is surgically operated as shown in FIG. 2, since there is a region E in which the inner circumference of the opening of the branch blood vessel 2 cannot be completely surrounded, a treatment effect cannot be obtained. In addition, when the stent S is surgically operated as shown in FIG. 3, blood flow disturbance may occur due to the partial region C of the stent S disposed in the main blood vessel 1.

### SUMMARY OF THE INVENTION

The present invention is directed to an H-side branch stent capable of preventing re-stenosis of the opening of the branch blood vessel since the stent can completely support an inner circumference of an opening of a branch blood vessel in a circumferential direction, preventing blood flow disturbance because stents are not densely concentrated to a boundary between a main blood vessel and the branch blood vessel and a partial region of the stent do not project into the main blood vessel, and completely adhering the stent to an inner wall of the main blood vessel with no gap.

In addition, the present invention is also directed to an H-side branch stent capable of treating a patient through a method the same as or similar to a conventional percutaneous coronary intervention (PCI), without a burden caused by a new operation method.

One aspect of the present invention provides an H-side branch stent including: a first stent having a plurality of cells and a hollow cylindrical shape; at least one bridge having one end connected to a distal end of the first stent; and a second stent having a front end connected to the other end of the bridge, and having a plurality of cells and a hollow cylindrical shape.

Another aspect of the present invention provides an H-side branch stent including: a first stent having a plurality of cells, a linear cutout part formed at an upper end in a longitudinal direction thereof, and a hollow cylindrical shape, which is expandable; at least one bridge having one end connected to a distal end of the first stent; and a second stent having a front end connected to the other end of the bridge, and having a plurality of cells and a hollow cylindrical shape.

Still another aspect of the present invention provides an H-side branch stent including: a first stent coated with a drug and having a plurality of cells and a hollow cylindrical shape; at least one bridge having one end connected to a distal end of the first stent; and a second stent coated with a drug and having a front end connected to the other end of the bridge, a plurality of cells and a hollow cylindrical shape.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 is a concept view showing a lesion B of a branch of the coronary artery;

FIGS. 2 and 3 are concept views showing a surgically operated state of a conventional stent used in a lesion of a branch of the coronary artery;

FIG. 4 is a perspective view showing an H-side branch stent in accordance with an exemplary embodiment of the present invention;

FIG. 5 is a detailed plan view showing a spread state of the H-side branch stent shown in FIG. 4;

FIG. 6 is a main side view of the H-side branch stent in accordance with an exemplary embodiment of the present invention;

FIG. 7 is a cross-sectional view showing a state in which the H-side branch stent shown in FIG. 4 is surgically operated;

FIG. 8 is a main side view of the H-side branch stent with no linear cutout part in accordance with the present invention;

FIG. 9 is a concept view showing a method of operating the H-side branch stent shown in FIG. 8 in a main blood vessel;

FIG. 10 is a main side view showing the H-side branch stent with a linear cutout part in accordance with the present invention;

FIG. 11 is a concept view showing a method of operating the H-side branch stent shown in FIG. 10 in the main blood vessel;

FIGS. 12 and 13 are concept views showing an operation process using the H-side branch stent in accordance with an exemplary embodiment of the present invention; and

FIG. 14 is a concept view of a state in which the H-side branch stent shown in FIG. 1 is surrounded by a dual balloon catheter.

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

With reference to the appended drawings, exemplary embodiments of the present invention will be described in detail below. To aid in understanding the present invention, like numbers refer to like elements throughout the description of the figures, and the description of the same elements will be not reiterated.

FIG. 4 is a perspective view showing an H-side branch stent in accordance with an exemplary embodiment of the present invention, FIG. 5 is a detailed plan view showing a spread state of the H-side branch stent shown in FIG. 4,

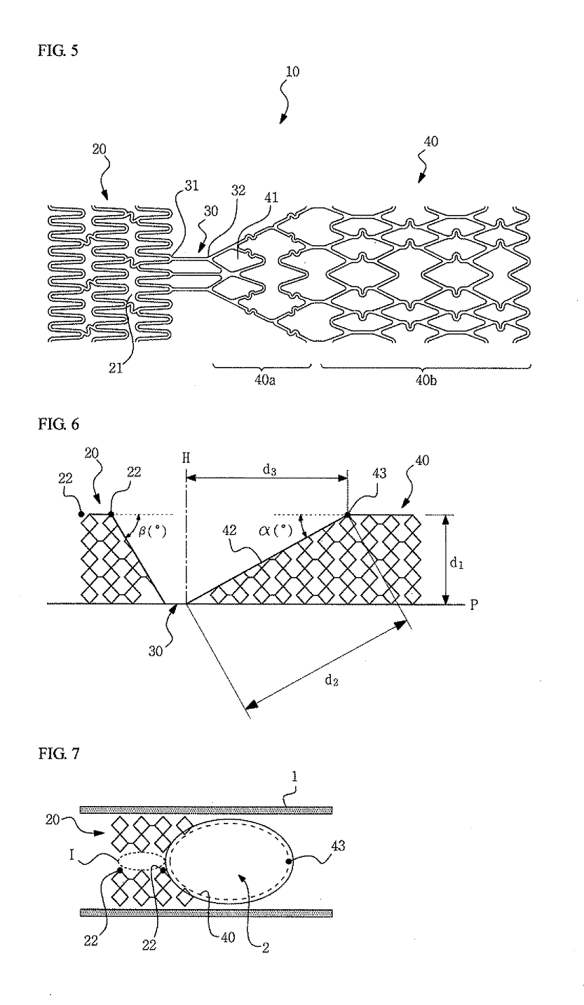

FIG. 6 is a main side view of the H-side branch stent in accordance with an exemplary embodiment of the present invention, and FIG. 7 is a cross-sectional view showing a state in which the H-side branch stent shown in FIG. 4 is surgically operated.

An H-side branch stent 10 in accordance with an exemplary embodiment of the present invention includes a first stent 20 having a plurality of cells 21 and a hollow cylindrical shape, one or more bridges 30 having one ends 31 connected to a distal end of the first stent 20, and a second stent 40 having a front end connected to the other end 32 of the bridge 30, and having a plurality of cells 41 and a hollow cylindrical shape.

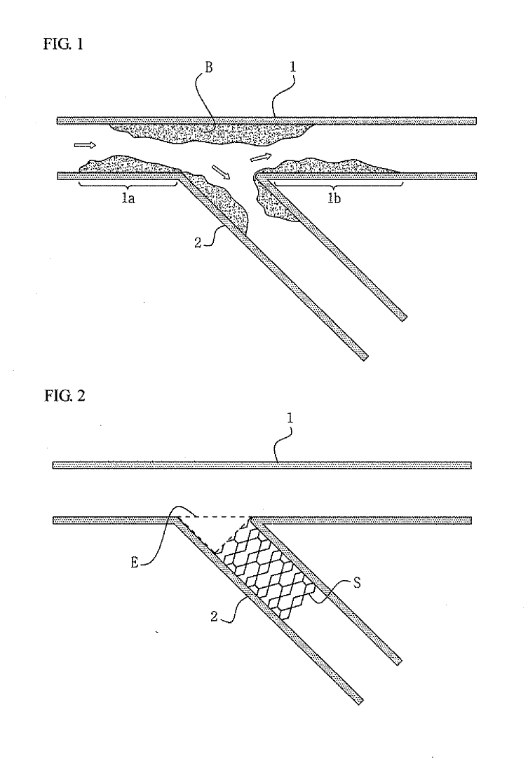

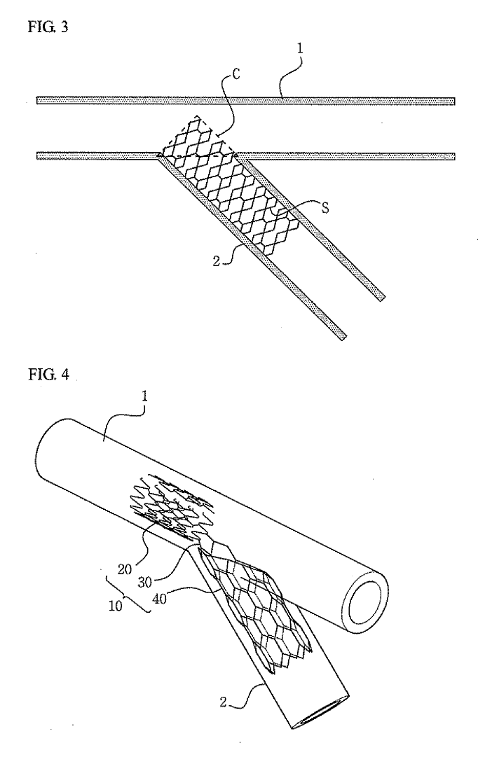

In the specification, a region which a blood flow approaches relatively in a flow direction of the blood flow (see FIG. 1) is referred to as a front end of a component and a region from which the blood flow goes away in the flow direction is referred to as a rear end of the component.

The first stent 20 corresponds to an inner wall of a main blood vessel 1 upon the surgical operation, and the second stent 40 corresponds to an inner wall of the branch blood vessel 2. Each of the stents 20 and 40 may be formed of at least one selected from the group consisting of stainless steel, cobalt, titanium, platinum, and an alloy thereof, which have a predetermined strength and resiliency, and may have a plurality of cells 21 or 41 so that the stent can be radially expanded in a cylindrical shape.

While sizes and cross-sectional shapes of the cells 21 and 41 may be determined according to an expansion level required in consideration of diameters of the main blood vessel and the branch blood vessel, the cells 41 formed at a front end 40a of the second stent 40 may have a diamond-like cross-section, or may be formed of a single open cell or a plurality of closed cells to have peaks and valleys. In addition, as shown in FIG. 5, the cells 21 of the first stent 20 may be formed by disposing corrugated parts formed of peaks and valleys adjacent to each other and connecting the two adjacent corrugated parts in a linear shape. At this time, cell density and linear connection strength may be determined a required expansion level.

Here, the front end 40a of the second stent 40 may be inclined toward the bridge 30, the distal end of the first stent 20 may be inclined toward the bridge 30, and the distal end of the first stent 20 and the front end of the second stent 40 may have an asymmetric “V” shape with respect to the bridge.

Meanwhile, an inclination angle a of the front end 40a of the second stent 40 is determined according to an angle of the branch blood vessel branched from the main blood vessel and a diameter of the branch blood vessel. While the inclination angle a may be smaller than a branch angle of a real blood vessel by about 1° to 3°, the inclination angle is not limited thereto but may be, for example, 20° to 120°.

Referring to FIG. 6, when a diameter d1 of the second stent 40 corresponds to a diameter of the branch blood vessel, a diameter d2 of the opening of the inclined opening and a length d3 of the inclined front end can be determined according to Pythagoras' theorem in which, when any two diameters are determined, the other one diameter is determined. Therefore, the inclined angle a and the other one diameter can be used to determine the other two diameters.

When the inclination angle α is 30° to 60° and the diameter of each of the stents 20 and 40 is 2 mm to 5 mm, the diameter d2 of the opening of the front end 40a of the second stent 40 may be 2 mm to 10 mm and the length d3 of the front end 40a of the second stent 40 may be 1 mm to 10 mm.

The length of the first stent 20 may be 3 mm to 15 mm. When the first stent 20 is shorter or longer than the length, unexpected complications may occur after the surgical operation, and when the length becomes longer, a gap may occur between the inner wall of the main blood vessel and the first stent by the longer extent.

In addition, the diameter of each of the stents 20 and 40 may be 2.5 mm to 5 mm in consideration of the diameters of the main blood vessel and the branch blood vessel, and the entire length of the first stent 20, the bridge 30 and the second stent 40 may be 8 mm to 30 mm.

The bridge 30 connecting the first stent 20 and the second stent 40 may be a plurality of linear bridges, may be formed of a metal material having a predetermined resiliency and strength to be bent, and may connect a lower end of the first stent to a lower end of the second stent. The bridge 30 provides the entire structural stabilization of the first stent 20 and the second stent 40 and contributes to technical improvement of delivery of the H-side branch stent 10.

Describing the stent with respect to a horizontal line P and a vertical line H passing through the bridge with reference to FIG. 6, the horizontal line H, the vertical line H and the diameter of the inclined opening of the front end of the second stent form substantially a right-angled triangle, and at this time, a region through which the horizontal line P passes becomes a lower end of each of the stents 20 and 40. Hereinafter, the region of each of the stents 20 and 40 through which the horizontal line P passes is referred to as the lower end, and an upper region of the opening is referred to as the upper end.

Meanwhile, the H-side branch stent 10 in accordance with an exemplary embodiment of the present invention may further include a marker 43 formed at a bending point of the front end 40a of the second stent 40, and a plurality of markers formed at the upper end of the first stent in a longitudinal direction thereof.

Referring to FIGS. 6 and 7, the markers 43 and 22, serving to precisely position the stent 10 in the main blood vessel and the branch blood vessel, may be Ruined of a metal or resin, through which radiation cannot penetrate, for example, at least one selected from the group consisting of stainless steel, gold and platinum. The markers 43 and 22 may be formed on the same line, and two adjacent markers 22 of the first stent 20 may have a gap of 0.5 mm to 5 mm, preferably, about 1 mm.

Meanwhile, the H-side branch stent 1 in accordance with an exemplary embodiment of the present invention may have a linear cutout part I formed at an upper end of the first stent 20 in a longitudinal direction thereof. The linear cutout part I is configured to provide a partially open cylindrical structure to the first stent 20 such that the diameter of the first stent 20 can be adjusted.

The linear cutout part 1 has a width of 10 mm or less. When the width is larger than 10 mm, a wrapping force of a balloon catheter for transporting the stent may be weakened to cause separation of the stent.

The first stent 20 performs important functions of safely transporting the H-side branch stent 10, preventing separation of the stent 10, preventing damage to the stent 10, and precisely positioning the second stent 40 in the branch blood vessel. Accordingly, the distal end of the first stent may be inclined toward the bridge 30 to a predetermined angle β.

In addition, the H-side branch stent 10 in accordance with an exemplary embodiment of the present invention may further include a drug coated on the first stent 20 and the second stent 40.

Here, the drug may be at least one selected from the group consisting of paclitaxel, sirolimus, biolimus, everolimus, zotalimus, tacrolimus, deforolimus and novelimus.

Hereinafter, a method of positioning the H-side branch stent 10 having the above-mentioned structure in the main blood vessel and the branch blood vessel will be described with reference to the accompanying drawings.

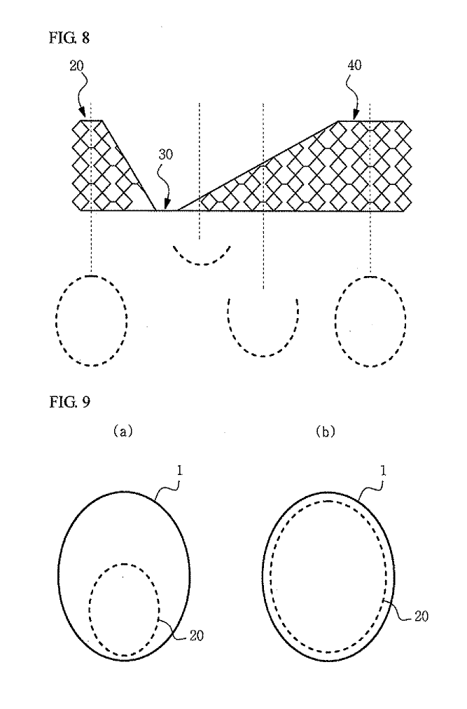

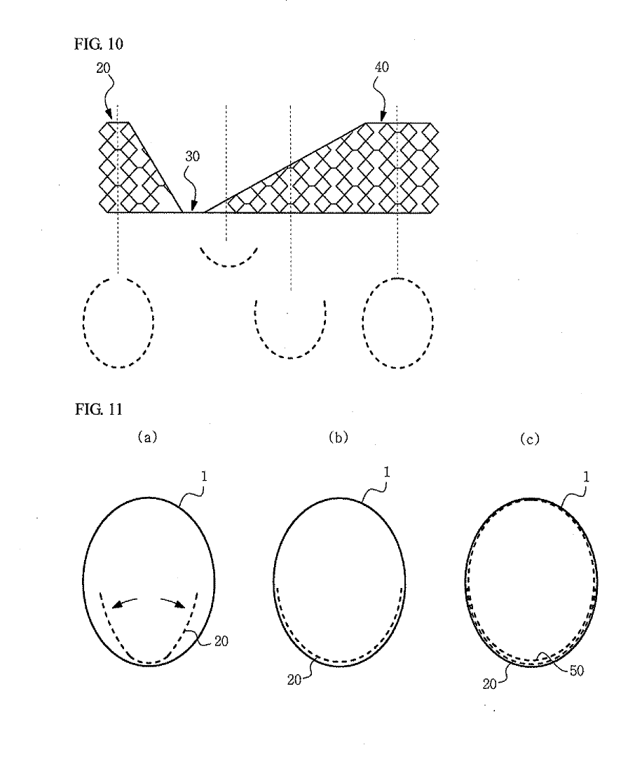

FIG. 8 is a main side view of the H-side branch stent with no linear cutout part in accordance with the present invention, FIG. 9 is a concept view showing a method of operating the H-side branch stent shown in FIG. 8 in a main blood vessel, FIG. 10 is a main side view showing the H-side branch stent with a linear cutout part in accordance with the present invention, and FIG. 11 is a concept view showing a method of operating the H-side branch stent shown in FIG. 10 in the main blood vessel.

In order to operate an ideal stent surgery for treatment of stenosis generated in the opening of the branch blood vessel 2, first, the opening of the branch blood vessel 2 must be supported with completely surrounded by the stent, second, dense concentration of the stent in a boundary between the main blood vessel and the branch blood vessel must be prevented, and finally, the stent disposed in the main blood vessel must be adhered to an inner wall of the main blood vessel with no gap.

For this, in the H-side branch stent 10 in accordance with the present invention, the opening is formed at the front end of the second stent 40 such that the inner wall of the branch blood vessel 2 can be supported with surrounded by the second stent 40, and the bridge 30 is disposed at the boundary between the main blood vessel 1 and the branch blood vessel 2 to prevent the dense concentration of the stent between the main blood vessel 1 and the branch blood vessel 2.

Referring to FIGS. 8 and 9, the first stent 20 has a cell structure to enable adjustment of the diameter. Here, the diameter of the first stent can be expanded by pressing the first stent outward in a radial direction. When the first stent 20 is disposed in the main blood vessel 1, the stent 20 can be expanded in the radial direction so that the first stent 20 can be completely adhered to the inner wall (partial region) of the main blood vessel 1.

Unlike this, referring to FIGS. 10 and 11, since the first stent 20 has a linear cutout part formed at the upper end thereof, the diameter of the first stent 20 can be adjusted. Here, the diameter of the first stent 20 can be expanded by pressing the first stent 20 in the radial direction. When the first stent 20 is disposed in the main blood vessel 1, the stent 20 can be expanded in the radial direction so that the first stent 20 can be completely adhered to the inner wall (partial region) of the main blood vessel 1.

After that, a new stent 50 may be inserted into the main blood vessel to complete treatment of the lesion of the branch blood vessel.

The operation method using the H-side branch stent 10 in accordance with an exemplary embodiment of the present invention is similar to the PCI in technical aspects.

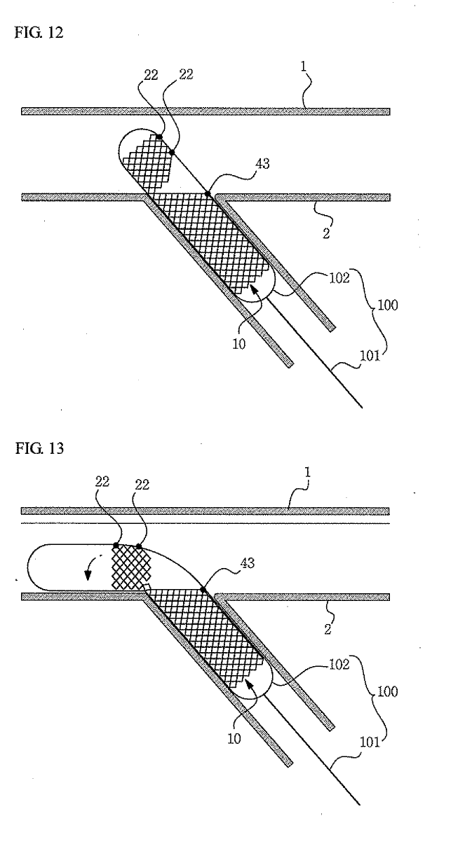

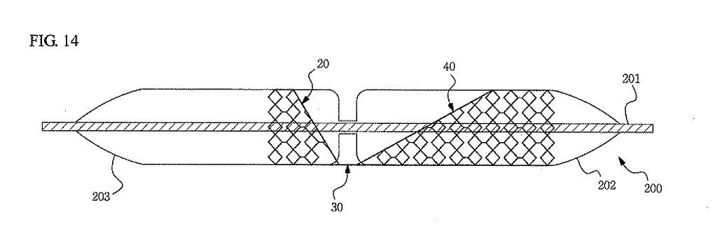

That is, referring to FIGS. 12 and 13, after inserting a guide wire 101 into the main blood vessel and the branch blood vessel, in a state in which the H-side branch stent 10 is accommodated in a balloon 102 along the guide wire 101, the balloon catheter 100 is moved toward the main blood vessel 1 and the branch blood vessel 2 to expand the severe stenosis area, obtaining a sufficient space to allow easy delivery of the stent 10 to the disease area.

Next, the H-side branch stent 10 corresponding to the diameter and branch angle of the branch blood vessel 2 is selected and disposed around the lesion through the balloon catheter 100.

Next, the second stent is precisely positioned in the opening of the branch blood vessel 2 with reference to the markers 22 and 43, and the first stent is pressed in the radial direction to be expanded and adhered to the inner wall of the main blood vessel.

Finally, as described with reference to FIG. 11 a new stent is disposed at the inner wall of the main blood vessel, and balloon dilatation is performed on the main blood vessel 1 and the branch blood vessel 2, completing treatment the lesion of the branch blood vessel.

Meanwhile, unlike this, as described with reference to FIG. 9, the first stent 20 can be expanded to be adhered to the inner wall of the main blood vessel 1 without use of the new stent.

FIG. 14 is a concept view of a state in which the H-side branch stent shown in FIG. 1 is surrounded by a dual balloon catheter 200. The dual balloon catheter 200 includes a balloon part including a front balloon 203 and a rear balloon 202, and a center shaft 201, so that the catheter 200 can be bent according to a curve of the blood vessel.

Here, the first stent 20 may be disposed in the front balloon 203 and the second stent 40 may be disposed in the rear balloon 202, and thus, the dual balloon catheter 200 may be bent at the bridge 30 of the stent.

Meanwhile, the stent of the present invention as described above may be used to the entire blood vessel of the body as well as the coronary artery. In particular, the stent may be used in blood vessels in the brain or liver, or the billary tract.

As can be seen from the foregoing, an H-side branch stent in accordance with the present invention can prevent re-stenosis of the opening of the branch blood vessel since the stent can completely support an inner circumference of an opening of a branch blood vessel in a circumferential direction, and prevent blood flow disturbance because stents are not densely concentrated to a boundary between a main blood vessel and the branch blood vessel and a partial region of the stent do not project into the main blood vessel, and completely adhere the stent to an inner wall of the main blood vessel with no gap.

In addition, an H-side branch stent in accordance with the present invention can treat a patient through a method the same as or similar to a conventional percutaneous coronary intervention (PCI), without a burden caused by a new operation method.

While the invention has been shown and described with reference to certain exemplary embodiments thereof, it will be understood by those skilled in the art that various changes in form and details may be made therein without departing from the scope of the invention as defined by the appended claims.

What is claimed is:

1. An H-side branch stent comprising:
a first stent having a plurality of cells and a hollow cylindrical shape;
at least one bridge having one end connected to a distal end of the first stent; and
a second stent having a front end connected to the other end of the bridge, and having a plurality of cells and a hollow cylindrical shape.

2. An H-side branch stent comprising:
a first stent having a plurality of cells, a linear cutout part formed at an upper end in a longitudinal direction thereof, and a hollow cylindrical shape, which is expandable;
at least one bridge having one end connected to a distal end of the first stent; and
a second stent having a front end connected to the other end of the bridge, and having a plurality of cells and a hollow cylindrical shape.

3. The H-side branch stent according to claim 1 or 2, wherein the front end of the second stent is inclined toward the bridge.

4. The H-side branch stent according to claim 1 or 2, wherein the distal end of the first stent is inclined toward the bridge.

5. The H-side branch stent according to claim 3, wherein the distal end of the first stent and the front end of the second stent have an asymmetric “V” shape with respect to the bridge.

6. The H-side branch stent according to claim 3, wherein an inclination angle of the front end of the second stent is 20° to 120°.

7. The H-side branch stent according to claim 3, wherein a diameter of an inclined opening of the front end of the second stent is 2 mm to 10 mm.

8. The H-side branch stent according to claim 3, further comprising a marker formed at a bending point of the front end of the second stent.

9. The H-side branch stent according to claim 8, further comprising a plurality of markers formed at an upper end of the first stent in a longitudinal direction thereof.

10. The H-side branch stent according to claim 9, wherein a gap between the two adjacent markers is 0.5 mm to 5 mm.

11. The H-side branch stent according to claim 9, wherein the markers are formed on the same line.

12. The H-side branch stent according to claim 9, wherein the markers are formed of a metal or resin through which radiation cannot pass.

13. The H-side branch stent according to claim 12, wherein the marker is formed of at least one selected from the group consisting of stainless steel, gold and platinum.

14. The H-side branch stent according to claim 1 or 2, wherein the bridge connects a lower end of the first stent and a lower end of the second stent.

15. The H-side branch stent according to claim 1 or 2, wherein the stent is formed of at least one selected from the group consisting of stainless steel, cobalt, titanium, platinum, and an alloy thereof.

16. The H-side branch stent according to claim 3, wherein the front end of the second stent has a length of 1 mm to 10 mm.

17. The H-side branch stent according to claim 3, wherein the cells formed at the front end of the second stent have a diamond-like cross-section.

18. The H-side branch stent according to claim 1 or 2, wherein the first stent has a length of 3 mm to 15 mm.

19. The H-side branch stent according to claim 2, wherein the linear cutout part has a width of 10 mm or less.

20. The H-side branch stent according to claim 1 or 2, wherein each of the stents has a diameter of 2 mm to 5 mm.

21. The H-side branch stent according to claim 1 or 2, wherein the entire length of the first stent, the bridge and the second stent is 8 mm to 30 mm.

22. An H-side branch stent comprising:
a first stent coated with a drug and having a plurality of cells and a hollow cylindrical shape;
at least one bridge having one end connected to a distal end of the first stent; and
a second stent coated with a drug and having a front end connected to the other end of the bridge, a plurality of cells and a hollow cylindrical shape.

23. The H-side branch stent according to claim 22, wherein the drug is at least one selected from the group consisting of paclitaxel, sirolimus, biolimus, everolimus, zotalimus, tacrolimus, deforolimus and novelimus.
